(19) United States
(12) Patent Application Publication
(10) Pub. No.: US 2022/0218243 A1
(43) Pub. Date: Jul. 14, 2022

(54) DEVICE and METHOD FOR MEASURING LEVEL OF CHLOROPHYLL IN BODY

(71) Applicant: Adam M. HASSE, Driftwood, TX (US)

(72) Inventor: Adam M. HASSE, Driftwood, TX (US)

(21) Appl. No.: 17/706,412

(22) Filed: Mar. 28, 2022

Related U.S. Application Data
(63) Continuation-in-part of application No. 16/164,019, filed on Oct. 18, 2018, now Pat. No. 11,304,931.
(60) Provisional application No. 62/584,971, filed on Nov. 13, 2017.

(51) Int. Cl. A61B 5/145; A61B 5/00

(52) U.S. Cl. CPC A61B 5/14546; A61B 5/742; A61B 5/0071

(57) ABSTRACT

A consumer electronic device and method for measuring the level of chlorophyll in a human subject. The device comprises a light source that exposes the subject to light to induce fluorescence of the chlorophyll, and a photometer that measures the level of fluorescing chlorophyll. The device may measure the level of chlorophyll without taking a blood or tissue sample, and may be a wearable device. The device may have at least one filter or surface that blocks light of undesired wavelengths or polarity, or selects light of desired wavelengths or polarity. The device may store, communicate, display or use the measured level of chlorophyll over a period of time.

### CROSS-REFERENCE TO RELATED APPLICATIONS

This application claims the benefit of U.S. Provisional Application No. 62/584,971, filed on Nov. 13, 2017, and U.S. application Ser. No. 16/164,019, filed Oct. 18, 2018, the disclosures of which are incorporated by reference in their entirety.

### FIELD OF THE INVENTION

The invention relates to devices and methods for measuring the level of chlorophyll in a human subject. More specifically, the invention relates to consumer electronic devices and methods comprising a light source that exposes the subject to light to induce fluorescence of the chlorophyll, and a photometer that measures the level of fluorescing chlorophyll. The device may measure the level of chlorophyll without taking a blood or tissue sample from the subject.

### BACKGROUND OF THE INVENTION

Modern dietary habits may result in significant nutritional shortfalls. There is long-standing consensus that regular consumption of chlorophyll-rich vegetables, such as dark green leafy vegetables, provides numerous health benefits in humans. Per calorie, green leafy vegetables are dense sources of potassium, fiber, folate, and other desirable phytochemical compounds. The vast majority of adults fail to eat the recommended amount of green leafy vegetables. There is a clear need for convenient and efficacious devices and methods for increasing consumption of such vegetables in the population.

Nutrition researchers and epidemiologists need to accurately measure people's dietary habits. Metabolic ward studies where food is provided are expensive and may not capture real world dietary patterns. Inexpensive questionnaires and surveys may capture poor data quality and often do not reflect what people actually eat. A device that can conveniently, inexpensively, and noninvasively measure intake of chlorophyll-rich vegetables would reduce the cost and improve the quality of dietary data available to researchers.

U.S. Patent Publication 2014/0058224 A1, Gellermann et al., discloses a method for measuring and quantifying biological compounds. In the method, a first side of a sample is illuminated with a light source, light transmitted through the sample is detected from an opposite second side of the sample, and a result is obtained based on the detected light.

Electronic devices positioned on or close to the body that monitor biometric parameters are known. Such devices have been used to measure heart rate, blood oxygenation levels, temperature, galvanic skin conductance, and other biometric parameters. These devices may have light sources that illuminate a portion of the skin of the user. The biometric parameters can then be determined by monitoring the amount of light absorbed or scattered by the illuminated portion of skin.

U.S. Pat. No. 10,722,157 B2, Bower et al., discloses an apparatus and method comprising a light collector configured to collect ambient light and provide the collected light to the skin of a user, a photodetector configured to enable a biometric parameter to be monitored by detecting changes in the light absorbed by the skin, and an amplifier configured to amplify an output signal provided by the photodetector. The light collector is configured to filter the collected ambient light to increase the proportion of light within a selected range of wavelengths provided to the skin of the user relative to the collected ambient light.

### SUMMARY OF THE INVENTION

The present invention relates to a consumer electronic device for measuring the level of chlorophyll in a human subject, comprising: (a) a light source that exposes the subject to light having a wavelength of from about 335 nm to about 750 nm to induce fluorescence of the chlorophyll in the subject, and (b) a photometer that measures the level of fluorescing chlorophyll in the subject in a wavelength range of from about 620 nm to about 900 nm.

In one embodiment, the invention relates to consumer electronic device for measuring the level of chlorophyll in a human subject without taking a blood or tissue sample from the subject, comprising: (a) an LED light source that exposes the subject to light having a wavelength of from about 335 nm to about 750 nm to induce fluorescence of the chlorophyll in the subject, and (b) a photometer that measures the level of fluorescing chlorophyll in the subject in a wavelength range of from about 620 nm to about 900 nm.

In another embodiment, the invention relates to a method for measuring the level of chlorophyll in a human subject using a consumer electronic device, said method comprising the steps of: (a) exposing the subject to a light source that provides light having a wavelength of from about 335 nm to about 750 nm to induce fluorescence of the chlorophyll in the subject, and (b) measuring the level of fluorescing chlorophyll in the subject in a wavelength range of from about 620 nm to about 900 nm using a photometer.

### BRIEF DESCRIPTION OF THE DRAWING

FIG. 1 is a block diagram illustrating an example of a consumer electronic device of the invention for measuring the level of chlorophyll in a subject.

### DETAILED DESCRIPTION OF THE INVENTION

The specification and appended claims refer to particular devices, features and methods of the invention. The invention includes all combinations, uses and steps described herein. The invention is not limited to or by the description of embodiments or examples in the specification, and the terminology used for describing particular embodiments and examples does not limit the scope or breadth of the invention.

The present invention provides devices and methods useful for measuring the level of chlorophyll in a human subject. The invention's ability to conveniently and noninvasively measure chlorophyll levels in vivo may provide feedback and motivation to health-conscious people and enable them to track and improve their dietary quality. By measuring chlorophyll in vivo, one can closely associate a positive action (eating chlorophyll-rich vegetables) with a positive reward (increased chlorophyll levels measured in the body), thereby increasing the probability one will consume more chlorophyll-rich vegetables (positive reinforcement). The invention thus enables users to monitor and increase their consumption of chlorophyll-rich vegetables, which have been widely demonstrated to improve health outcomes. To help achieve this goal, it is useful to incorporate such a measuring device into a consumer electronic device, particularly a portable device that can easily be carried or moved by the user, such as a smartphone, smartwatch, smart bracelet, or the like. Such a device can be used to store and accumulate data over time and make the data easily communicated to the user and other interested parties, such as health professionals, researchers, insurance companies, or social media.

In addition to the above, chlorophyll likely has health promoting effects beyond just being a marker for other beneficial compounds present in chlorophyll-rich vegetables. As described in U.S. application Ser. No. 16/164,019, chlorophyll may be used to catalyze the regeneration of ubiquinol antioxidant in vivo. Also, many mutagenic and carcinogenic chemicals, such as benzene, have fiat ring-like geometry and can slip between the “rungs” of DNA and bind there. Chlorophyll is a so-called interceptor molecule that can bind to some of these mutagenic and carcinogenic chemicals, rendering them unable to disturb the operation of DNA. Chlorophyll is also known to concentrate in the mitochondria of mammals that consume a chlorophyll rich diet, and is speculated to play a role in enhancing the function of mitochondria.

As used herein, the term “chlorophyll” includes chlorophyll a, b, c1, c2, c3, d and f, chlorophyll metabolites, and/or synthetic modifications of chlorophyll and its metabolites. Chlorophyll metabolites useful herein include, but are not limited to, those described in “Qu, J. et al., Dietary Chlorophyll Metabolites Catalyze the Photoreduction of Plasma Ubiquinone, Photochemistry and Photobiology, 89: 310-313 (2013), incorporated herein by reference. A variety of chlorophyll metabolites, such as chlorophyllide-a, pheophytin-a, pheophorbide-a, methyl pheophorbide-a, 10-OH-pheophorbide-a, 10-OH-methyl pheophorbide-a, pyropheophorbide-a and methyl pyropheophorbide, may be present in the blood or body tissue. The above metabolites may be formed from chlorophyll by chemistry that normally takes place in the body. Synthetic modifications of chlorophyll and its metabolites may provide additional or different properties, for example, that extend the half-life, bioavailability or reactivity of chlorophyll compounds and metabolites in the body.

100161 Chlorophyll is found in green leafy vegetables, such as spinach, kale, cabbage, garden cress, bok choy, broccoli, and brussels sprouts, and sea vegetables such as edible algae and seaweed. Chlorophyll is an organic molecule sensitive to blue and red wavelengths of light that excels in capturing electromagnetic energy, some of which is released during fluorescence. When humans consume chlorophyll-rich foods, chlorophyll is absorbed into and circulates throughout the bloodstream and body tissues. Red light from an external source can penetrate into the body in sufficient quantity to stimulate chlorophyll in the blood and body tissues and induce fluorescence of the chlorophyll.

Chlorophyll from terrestrial plants (as opposed to seaweed/algae) typically comprises chlorophyll a and chlorophyll b. Plants grown in a high light environment typically bias more towards chlorophyll a production, while low light conditions bias a plant to produce more chlorophyll b. The bulk of vegetables for human consumption are grown on commercial farms in a high light environment and thus typically have about a 3:1 ratio of chlorophyll a to chlorophyll b. Thus, it may be desirable to select light wavelengths and filters biased more toward the chlorophyll a absorption and fluorescence spectrum.

Human tissue is relatively transparent to orange, red and near infrared wavelengths of light from about 590 nm to about 900 nm. In this wavelength range, light can penetrate into the body and scatter across depths up to about 100 mm. Chlorophyll absorbs light in this wavelength range, and also fluoresces at characteristic wavelengths in this window of relative tissue transparency. Thus, it is possible to induce fluorescence of chlorophyll in the body with a light source and then measure the level of light from the fluorescing chlorophyll with a photometer. Chlorophyll has a relatively high fluorescence quantum yield (the ratio of the number of photons emitted to the number of photons absorbed) for a large organic molecule, which allows it to fluoresce about 0.05-2% of the energy it absorbs. The overlapping wavelength properties (absorbance and fluorescence) of chlorophyll and human tissue transparency allows one to construct a device capable of measuring chlorophyll in vivo with a large signal to noise ratio. Wavelength ranges and filters can be selected to enhance the measurement of fluorescing chlorophyll. Because chlorophyll in vivo may be present in small quantities compared to other compounds in the bloodstream and body tissue, such as hemoglobin and blood glucose, a high signal to noise ratio is needed to produce a reliable and repeatable measurement of chlorophyll in vivo. This can be achieved in devices of the invention constructed with inexpensive, compact, commercially available semiconductor technology. In contrast, resonance Raman spectroscopy (RRS) requires expensive and bulky equipment to measure pigments in human tissue, and provides a very small output signal (e.g., about 1 in 10 million photons are inelastically scattered off the target molecules).

Increasing the signal to noise ratio of the measurement of chlorophyll fluorescence improves the accuracy and repeatability of the measurement. To this end, applying at least one filter or surface to or adjacent the light source and/or photometer allows one to selectively block light of undesired wavelengths or polarity from interacting with the photometer or stimulating other compounds in the body which may have competing fluorescent patterns. The device with the light source and photometer may be placed in close proximity to the skin to increase sensitivity of the measurement and block ambient light. The photometer and/or other sensors on the device may be used to measure ambient light and other parameters as part of the calibration of the device. The timing of the light source emission and photometer measurement may also be varied to improve measurement accuracy. For example, the photometer may first make a measurement with the light source off to produce a control signal of the ambient light. The light source may then be turned on while the photometer measures the experimental signal, which includes the photons generated by the fluorescing chlorophyll. Subtracting the control signal from the experimental signal may reduce confounding variables and produce a better measurement. In other embodiments, the light source may be modulated on/off in a specific or selected pattern, and/or the photometer may measure levels of fluorescing chlorophyll in a consistent spectral or temporal pattern, such as a ratio of fluorescence peaks, which may improve confidence in the measurement. The device may take measurements throughout the day and store that data to produce a datalog of chlorophyll levels over time and provide a means to communicate that dataset to the user and other parties.

The device of the invention is a portable, consumer electronic device, such as a computer or smartphone, or a wearable consumer electronic device, such as a smartwatch, Fitbit product, smart bracelet, or patch or sticker having at least one adhesive portion for attaching the device to the body or an article in contact with or proximity to the body, such as an item of clothing. In such a device, the components may be integrated into a single unit. In other examples, the device may be an LED-embedded wristband worn in close contact with the high blood flow in the wrist, or a neck band or collar with an LED in close proximity to the carotid arteries, or an LED-embedded vest or wrap worn across the abdomen to measure chlorophyll in the digestive tract. The device typically is placed on or near the skin of the subject, such as on a finger, hand, wrist, arm, neck, abdomen, or earlobe. The light source, typically an LED, and photometer, typically a photodiode, may be located adjacent to each other, e.g., coplanar or on the same side of the device. In a smartwatch or similar device, the light source or photometer may be located in a wrist strap and the other component located in the body of the device. The device may be used to measure the level of chlorophyll in a subject at a single time, at intervals of time, or for an extended period of time. Wearable devices may conveniently measure chlorophyll levels as the user goes about his or her day, and may be worn for extended periods of time without causing discomfort to the user. The device may have low average-power requirements so a reliable output signal can be provided over an extended period of time. In one embodiment, the device measures the level of chlorophyll in the subject without taking a blood or tissue sample from the subject.

The light source exposes the subject to light having a wavelength of from about 335 nm to about 750 nm, typically from about 430 nm to about 700 nm, more typically from about 590 nm to about 670 nm, for example, from about 600 nm to 649 nm, to induce fluorescence of the chlorophyll in the subject. The light may be provided in a narrow wavelength range optimized for particular conditions, or the light may have a broad wavelength range so long as sufficient light in the specified range is provided to induce fluorescence of the chlorophyll. The light generation and delivery may be provided by various methods. The light source may be a light emitting diode (LED), a light emitting diode array, a tungsten halogen lamp, a laser, or any other suitable light source, or combinations thereof. The light is typically provided by an efficient light source, such as an LED light source, which may be included in a portable device designed to be worn by the subject. For example, the light may be provided by one or more LEDs in a device attached to or strapped onto the body or embedded in clothing.

The photometer measures the level of fluorescing chlorophyll in the subject in a wavelength range of from about 620 nm to about 900 nm, typically from about 640 nm to about 850 nm, for example, from 650 nm to about 820 nm, and is sensitive to the fluorescing chlorophyll at these wavelengths. The photometer may detect light transmitted from the fluorescing chlorophyll in spectrally resolved detection configurations or at strategically chosen discrete wavelengths. The photometer may include a spectrograph/charge coupled (CCD) or CMOS detector configuration, a photomultiplier tube, a photodiode detector, an avalanche photodiode, and/or other optical detectors, including a spatially integrating optical detector. Avalanche photodiodes containing graphene may be particularly useful in consumer electronic devices because of their reduced size and cost, and increased sensitivity and durability.

The photometer may include a collection module, a spectral selection module, and/or a light detection module. The collection module may collect the light from the fluorescing chlorophyll. The collection module may include a charge coupled device (CCD) camera, a CCD array, a CMOS detector, a photomultiplier tube, a photodiode detector, an avalanche photodiode, and/or other optical detectors. A CCD array is an array of pixels that detects light intensities and wavelengths corresponding with the pixels. In some configurations, the collection module may include a spatially integrating optical detector. The spectral selection module may filter out unwanted wavelengths of light.

In some examples, the photometer may comprise a photodiode, a phototransistor, a light dependent resistor, a photodetector constructed with organic light sensitive materials, a graphene FET based photodetector, or any other suitable means. The photometer may comprise highly sensitive material such as quantum dot material, semiconductor nanoparticles or any other material which is a strong absorber of light. The material used within the photometer may be selected to be optimized for light detection within selected wavelengths. In some examples, the device may comprise an array of photometers or a plurality of photometers. Using an array of photometers may improve the signal to noise ratio in the light collected by the array compared to the light collected by a single photometer. Using an array of photometers may also reduce the effects of motion-based artifacts as a result of relative motion between the photometer and the skin of the user.

The photometer may convert the detected light into an electronic signal, and may send the electronic signal to an acquisition, quantification and display module, which may analyze and quantify the electronic signal and display a result using suitable data acquisition and processing routines. The result may include the level of fluorescing chlorophyll in the subject in the desired wavelength range.

Determining the level of fluorescing chlorophyll in the subject may include processing the electronic signal from the photometer. Processing the electronic signal may include analyzing and/or visually displaying the signal on a monitor and/or other display. Processing the electronic signal from the photometer may further include converting the light signal into other digital and/or numerical formats. Data acquisition software may be used by the quantification and display module to determine the levels of fluorescing chlorophyll. For example, the quantification and display module may analyze, quantify and display the level of fluorescing chlorophyll. Additionally, the quantification and display module may compare concentration levels of fluorescing chlorophyll in the result to previous concentration levels of fluorescing chlorophyll in the subject to track changes in response to dietary changes or supplementation, and provide an indication of changes in the level of chlorophyll-rich vegetables consumed by the subject over time. In some configurations, the quantification and display module may be a computing device, such as a personal computer or may include other computing devices.

The photometer or array of photometers may be coupled to an amplifier. The output signal from the photometer may be provided as an input signal to the amplifier. The photometer and amplifier may be operationally coupled, and any number of components may be provided between the amplifier and the photodetector, including no intervening components. The amplifier may comprise any means which may be configured to receive the output signal from the photometer and amplify the signal to provide an amplified output signal. The amplifier may comprise a low power operational amplifier circuit. The amplifier may be arranged to provide a large gain. This may enable a very low power input signal to be used to obtain a useful output signal. The gain provided by the amplifier may be programmable. For example, the gain may be programmed to be dependent upon the intensity of the output signal or intensity of ambient light. In such cases the gain provided by the amplifier may be lower when the output signal has a high intensity and the gain may be higher when the output signal has a low intensity. This may enable the power consumption of the device to be minimized and improve the signal to noise ratio. The output signal may be provided to an output device, such as a display or other suitable device, so that a user or other party can view the obtained information relating to chlorophyll levels.

In one embodiment, the device comprises a light source that exposes a portion of the skin of the subject to light having a wavelength of from about 335 nm to about 750 nm to induce fluorescence of the chlorophyll in the subject; a photometer that measures the level of fluorescing chlorophyll in the subject in a wavelength range of from about 620 nm to about 900 nm; and an amplifier configured to amplify an output signal provided by the photometer. The device may be configured to block light of undesired wavelengths or polarity, increase light within a selected range of wavelengths to be provided to the subject, and/or increase the signal to noise ratio of the measured level of fluorescing chlorophyll.

In one embodiment, the wavelength provided by the light source is less than the wavelength of the fluorescing chlorophyll measured by the photometer. Since the wavelength of the output light is greater than the wavelength of the input light, there is a high degree of confidence that the photometer has measured the level of fluorescing chlorophyll in the subject. In one example, the light source exposes the subject to light having a wavelength of from about 600 nm to 649 nm to induce fluorescence of the chlorophyll in the subject, and the photometer measures the level of fluorescing chlorophyll in the subject in a wavelength range of from 650 nm to about 820 nm.

In another embodiment, the device further comprises at least one filter or surface that blocks light of undesired wavelengths or polarity, or selects light of desired wavelengths or polarity. For example, the filter or surface may block light of unwanted wavelengths generated by the light source, block unwanted ambient light from reaching the photometer, and/or block competing fluorescent light from other compounds, molecules or materials in the body. Alternatively, the filter or surface may select light of desired wavelengths generated by the light source to induce fluorescence of the chlorophyll in the subject.

In some embodiments, the device, or the light source or photometer components thereof, may comprise one or more filters or surfaces configured to spectrally filter or block light of undesired wavelengths or polarity. The one or more filters or surfaces may comprise one or more diffraction gratings, dichroic filters, dichroic mirrors or any other suitable means. The one or more filters or surfaces may be configured to allow light within selected wavelength ranges to pass through. The one or more filters or surfaces may be configured to prevent light not in selected wavelength ranges from passing through.

Photometers, acquisition, quantification and display modules, amplifiers, and filters or surfaces that blocks light of undesired wavelengths or polarity or select light of desired wavelengths or polarity, suitable for use in the present invention are disclosed in U.S. Patent Publication 2014/0058224 A1 and U.S. Pat. No. 10,722,157 B2, both incorporated herein by reference.

In another embodiment, the device may utilize polarized light to distinguish wavelengths from the light source versus wavelengths from the fluorescing chlorophyll. For example, one may place a polarizing filter over the light source to orient emitted light into a particular direction or spin. As this input light travels through blood or tissue, it will either be scattered, reflected, or absorbed. Scattered and reflected light will maintain a level of polarization, but light that is absorbed and fluoresced by chlorophyll will not be polarized. If a second polarizing filter is placed over the photometer, the partially polarized scattered and reflected light from the light source may be dampened relative to the unpolarized light emitted by fluorescing chlorophyll. In this way, one may optimize the wavelength spectrum of the light source to match the absorbance patterns of chlorophyll without exposing the photometer to input from the light source and reducing the signal to noise ratio of the measurement. However, using polarized light to improve the signal to noise ratio causes an overall damping of light intensity because unpolarized light is composed alight oriented in all directions. if roughly half the light has a component vector in any given direction, a polarizing filter may block the available light by a factor of two. If two polarizing filters are used in the device, the light source would need to emit about four times as much light to achieve the same signal strength in the photometer.

In another embodiment, the device stores, communicates, displays or uses the measured level of chlorophyll in the subject over a period of time. For example, the measured level of chlorophyll may be stored, communicated or displayed for use by the subject, or transmitted to nutrition researchers, epidemiologists, health care providers, insurers, or social media to help evaluate or improve the subject's dietary habits, or to encourage the subject to increase or adjust ingestion of chlorophyll-rich vegetables to a desired level over time.

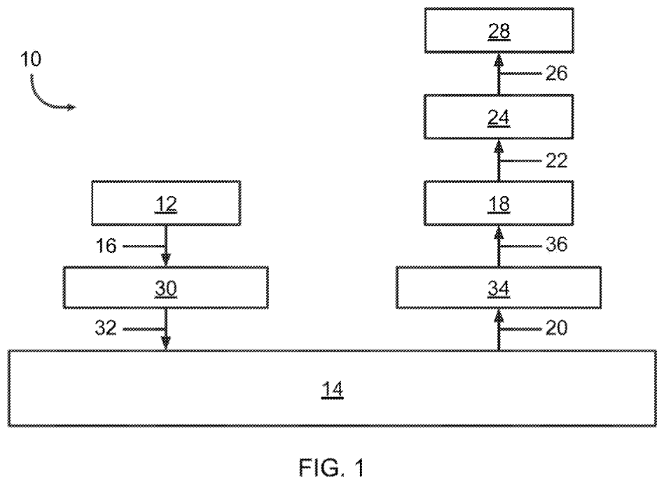

FIG. 1 is a block diagram illustrating a consumer electronic device 10 of the invention for measuring the level of chlorophyll in a human subject. The device 10 includes a light source 12 that exposes a portion of the skin 14 of the subject to light 16 having a wavelength of from about 335 nm to about 750 nm to induce fluorescence of the chlorophyll in the subject. Device 10 also includes a photometer 18 that measures the level of fluorescing chlorophyll light 20 in a wavelength range of from about 620 nm to about 900 nm. Photometer 18 is coupled to an amplifier 24 such that the signal 22 from the photometer is inputted to the amplifier. The amplified signal 26 from the amplifier is sent to a quantification and display module 28, which analyzes and quantifies the signal and displays a result using suitable data acquisition and processing routines. Device 10 further comprises a filter 30 that receives the light 16 from the light source and filters the light 16 before transmitting filtered light 32 to the skin 14 of the subject. A second filter 34 receives the fluorescing chlorophyll light 2.0 and filters the light 20 before transmitting filtered light 36 to the photometer 18. The components of device 10 are combined in a single device, such as a wearable consumer electronic device.

While the present invention can be used to improve dietary habits by increasing consumption of chlorophyll-rich vegetables, if desired or as a calibration tool or step, chlorophyll may be provided to the subject in the form of a supplemental or therapeutic composition in any acceptable dosage form, including capsules, tablets, lozenges, troches, hard candies, powders, sprays, elixirs, syrups, suspensions or solutions, and topical compositions, such as disclosed in U.S. Pat. No. 7,435,725, incorporated herein by reference. Such dosage forms may be conveniently taken before, during or between meals. The chlorophyll composition may be provided in the form of chlorophyll in and/or its salts, which may be administered to a subject in amounts that provide a daily dosage between about 20 mg and about 500 mg, typically about 40 mg to about 400 mg, more typically about 60 mg to about 300 mg. Since chlorophyllin and its salts are water soluble, they may be better suited than other compositions for supplementation.

The time interval between light exposure and measurement of fluorescing chlorophyll may vary based upon the method of delivery of chlorophyll to the subject. For example, it may take about 15 minutes for a stand-alone liquid to leave the stomach and enter the small intestine where absorption occurs. If the chlorophyll-containing composition is a solid food or is mixed with solid food, it may take up to about 2 hours or more to leave the stomach and enter the intestines where absorption occurs to provide a measurable level of fluorescing chlorophyll in the bloodstream or body tissue. Since chlorophyll is lipid-soluble, ingestion with lipids may increase absorption of the chlorophyll in the subject.

It may be desirable for the light exposure and measurement of fluorescing chlorophyll to occur while the subject is exercising, or shortly after exercising, because the increased heart rate and blood flow may provide increased mixing and circulation of the chlorophyll in the body. During exercise, the blood is also brought closer to skin surfaces, which may increase the amount of light reaching the chlorophyll in the bloodstream. Because some wearable devices, such as a smartwatch, already measure heart rate and may have an accelerometer, it may be convenient to measure the level of chlorophyll during exercise when the blood is close to body surfaces. This may also result in a more accurate measurement of the level of fluorescing chlorophyll in the subject.

In one embodiment, the invention may estimate the level of chlorophyll in the bloodstream in real time without drawing blood samples to directly measure the chlorophyll level. Since chlorophyll fluoresces at unique wavelengths, for example, it emits at about 680 nm to about 750 nm (e.g., 685 or 730 nm) when exposed to light having a wavelength of about 335 nm to about 360 nm (e.g., 355 nm) or about 457 nm, and the body is semi-transparent to this fluoresced wavelength, one can measure blood chlorophyll levels in real time using fluorescence spectroscopy and a sensitive photometer and correlate this to actual chlorophyll levels via a scaling factor determined by experimental observation. Thus, in another embodiment, the invention provides a method for estimating the level of chlorophyll in the bloodstream, comprising the steps of: (a) exposing the subject's skin to light to induce fluorescence of the chlorophyll, (b) measuring the level of fluorescing chlorophyll in the bloodstream using fluorescence spectroscopy, and (c) correlating the level of fluorescing chlorophyll using a predetermined scaling factor. The scaling factor may be experimentally determined by measuring chlorophyll blood levels before and after exposure to known quantities of chlorophyll and light having a wavelength as described above on a control group of representative subjects. The method of estimating the level of chlorophyll in the bloodstream may be accomplished without drawing a blood sample from the subject. The level of chlorophyll in the bloodstream of the subject can then be adjusted upward to a desired level by adjusting the intake of the chlorophyll composition, as described above.

Where a range of values is provided, it is understood that each intervening value between the upper and lower limit of that range and any other stated or intervening value in that range is encompassed within the invention. The upper and lower limits of these smaller ranges may independently be included in the smaller ranges, and is also encompassed within the invention, subject to any specifically excluded limit in the stated range. Where the stated range includes one or both of the limits, ranges excluding either or both of those included limits are also included in the invention.

It should be apparent to those skilled in the art that modifications of the present invention besides those described are possible without departing from the inventive concepts. The inventive subject matter, therefore, is not restricted except as stated in the disclosure.

In interpreting the disclosure, all terms should be interpreted in the broadest possible manner consistent with the context. In particular, the terms “comprise” and “comprising” should be interpreted as referring to elements, components, or steps in a nonexclusive manner, indicating that the referenced elements, components, or steps may be present, or utilized, or combined with other elements, components, or steps that are not expressly referenced.

Where reference is made to a method comprising two or more defined steps, the steps can be carried out in any order or simultaneously (except where the context excludes that possibility), and the method can include one or more other steps which are carried out before any of the defined steps, between two of the defined steps, or after all the defined steps (except where the context excludes that possibility).

What is claimed is:

1. A consumer electronic device for measuring the level of chlorophyll in a human subject, comprising:
(a) a light source that exposes the subject to light having a wavelength of from about 335 nm to about 750 nm to induce fluorescence of the chlorophyll in the subject, and
(b) a photometer that measures the level of fluorescing chlorophyll in the subject in a wavelength range of from about 620 nm to about 900 nm.

2. The device of claim 1 that measures the level of chlorophyll in the subject without taking a blood or tissue sample from the subject.

3. The device of claim 1 wherein the light source is an LED light source.

4. The device of claim 1 wherein the wavelength provided by the light source is less than the wavelength of the fluorescing chlorophyll measured by the photometer.

5. The device of claim 1 where the light provided by the light source has a wavelength of from about 600 nm to 649 nm, and the photometer measures the level of fluorescing chlorophyll in a wavelength range of from 650 nm to about 820 nm.

6. The device of claim 1 that is a wearable consumer electronic device.

7. The device of claim 1 comprising an amplifier configured to amplify an output signal provided by the photometer.

8. The device of claim 1 further comprising at least one filter or surface that blocks light of undesired wavelengths or polarity, or selects light of desired wavelengths or polarity.

9. The device of claim 1 that stores, communicates, displays or uses the measured level of chlorophyll in the subject over a period of time.

10. A consumer electronic device for measuring the level of chlorophyll in a human subject without taking a blood or tissue sample from the subject, comprising:
(a) an LED light source that exposes the subject to light having a wavelength of from about 335 nm to about 750 nm to induce fluorescence of the chlorophyll in the subject, and
(b) a photometer that measures the level of fluorescing chlorophyll in the subject in a wavelength range of from about 620 nm to about 900 nm.

11. The device of claim 10 wherein the wavelength provided by the light source is less than the wavelength of the fluorescing chlorophyll measured by the photometer.

12. The device of claim 10 where the light provided by the light source has a wavelength of from about 600 nm to 649 nm, and the photometer measures the level of fluorescing chlorophyll in a wavelength range of from 650 nm to about 820 nm.

13. The device of claim 10 that is a wearable consumer electronic device.

14. The device of claim 10 comprising an amplifier configured to amplify an output signal provided by the photometer.

15. The device of claim 10 further comprising at least one filter or surface that blocks light of undesired wavelengths or polarity, or selects light of desired wavelengths or polarity.

16. The device of claim 10 that stores, communicates, displays or uses the measured level of chlorophyll in the subject over a period of time.

17. A method for measuring the level of chlorophyll in a human subject using a consumer electronic device, said method comprising the steps of:
(a) exposing the subject to a light source that provides light having a wavelength of from about 335 nm to about 750 nm to induce fluorescence of the chlorophyll in the subject, and
(b) measuring the level of fluorescing chlorophyll in the subject in a wavelength range of from about 620 nm to about 900 nm using a photometer.

18. The method of claim 17 accomplished without taking a blood or tissue sample from the subject.

19. The method of claim 17 where the light provided by the light source has a wavelength of from about 600 nm to 649 nm, and the photometer measures the level of fluorescing chlorophyll in a wavelength range of from 650 nm to about 820 nm.

20. The method of claim 17 where the device is a wearable device.

21. The method of claim 17 where the device has at least one filter or surface that blocks light of undesired wavelengths or polarity, or selects light of desired wavelengths or polarity.

22. The method of claim 17 where the device stores, communicates, displays or uses the measured level of chlorophyll in the subject over a period of time.
